(19) United States
(12) Patent Application Publication
(10) Pub. No.: US 2014/0326056 A1
(43) Pub. Date: Nov. 6, 2014

(54) Method for Quick Assessment of Osmolarity

(71) Applicant: D.E.S. DIAGNOSTICS LTD, Herzliya (IL)

(72) Inventor: Eran Eilat, Herzliya (IL)

(21) Appl. No.: 13/982,758

(22) PCT Filed: Nov. 29, 2012
(86) PCT No.: PCT/IL2012/000384

§ 371 Date: Jul. 31, 2013

Related U.S. Application Data
(60) Provisional application No. 61/564,925, filed on Nov. 30, 2011.

(51) Int. Cl. G01N 33/487

(52) U.S. Cl. CPC G01N 33/487
USPC 73/64.47

(57) ABSTRACT

An osmolarity measuring device for providing a semi-quantitative color measure of liquid's osmolarity, said device comprising: a. none or more coloring agents; b. none or more swelling agents; c. none or more solutions; d. a vesicle with a continuous integument, containing within said integument at least one of a group consisting of said coloring agent, said swelling agent and said solution; and e. a support of absorbent material impregnated in at least one area with a plurality of said vesicles; wherein the apparent color of said area alters if the osmolarity of said liquid is within a pre-determined range.

### FIELD OF THE INVENTION

The present invention generally pertains to a system and method for quick assessment of osmolarity.

### BACKGROUND OF THE INVENTION

Many membranes are permeable only to water; such permeability allows equality of osmolarity of both sides of the membrane, through the movement of water to ensure equilibrium. Hence, water will move from the lower osmolarity region to the higher osmolarity region.

A description of the importance of tear osmolarity is taught in U.S. Pat. No. 7,129,717: Systems and methods for measuring tear film osmolarity:
“Tears fulfill an essential role in maintaining ocular surface integrity, protecting against microbial challenge, and preserving visual acuity. These functions in turn, are critically dependent upon the composition and stability of the tear film structure, which includes an underlying mucin foundation, a middle aqueous component, and an overlying lipid layer. Disruption, deficiency, or absence of the tear film can severely impact the eye.
An increased salt concentration (osmolarity) of the human tear film has been identified as the underlying causative mechanism for all types of dry eye. Chronically heightened osmolarity is tied to post-LASIK complications, keratoconjunctivitis sicca, and contact-lens induced dry eye. While its usefulness as a marker of tear film health is evident, the ability to rapidly measure tear osmolarity has eluded science for decades. If it were possible to measure and, more importantly, monitor the relative tear film osmolarity over time, the quality of health care that could be provided to dry eye patients would increase dramatically. Personalized data about the state of one's disease would allow physicians to develop new therapies, alter prescriptions, and modulate the type of products used in a timely fashion.
For example, the day-to-day response of the tear film is of particular interest when challenged by the deleterious effects of contact lens wear. Irritation caused by contact lenses may lead to an inflammatory condition that exacerbates dry eye symptoms and causes lens wearers considerable discomfort. Often, when getting fitted for a new prescription, a patient must try several different types of lenses before finding a brand that is optimized for their individual ocular surface. The fitting process is largely trial and error because the clinician lacks the ability to track the patient's response over time. Further, because deleterious inflammatory effects may take a few hours to days to occur, it is not feasible to gather this data while the patient is sitting in a clinician's office.
The same constraints hold for monitoring the efficacy of dry eye therapeutics, whether pharmaceutical, tear replacement, or mechanical, i.e. goggles, punctal plugs, etc. Physicians have no means to collect, or correlate patient data, i.e. race, age, gender, medications taken, etc. with signs and symptoms over time. Likewise, scientists would benefit greatly from this data when developing new techniques to treat the disease.
Prevalent technologies require collection of tears by gently touching a glass capillary to the lower lid. However, such technologies are not amenable to “at home” testing. Furthermore, the clinical equipment necessary to analyze the tear film is far too expensive for personal monitoring of tear film health.”

Examples of patents teaching other technologies for measuring tear film quality or tear quantity are U.S. Pat. No. 4,747,683, US20100085540A1, U.S. Pat. No. 7,771,353, US20090168019A1, WO2008 156883A3, US20080273171A1, EPI844703AI, JP2006314651A2, JP2004242713A2, JP08052112A2, JP06277179A2, U.S. Pat. No. 7,988,294, U.S. Pat. No. 7,809,417, U.S. Pat. No. 7,758,190 and others. All suffer from the defects described above.

Osmolarity of saliva also depends on the patient's state of health, although there is more variability in the osmolarity of saliva in normal subjects than there is with the osmolarity of tears. High osmolarity of saliva may indicate damage to the parotid duct, inflammation or allergy. It is also frequently seen in children with cerebral palsy, where it is correlated with impaired adequate hydration status. Since the possible hypohydration condition may be reflected in saliva fluid, which could compromise the protective function exerted by saliva, the earlier this condition is identified the greater the chances of administering preventive measures. Testing of saliva is also used to indicate possible dehydration in persons under stress, such as athletes or persons using protective clothing.

It is therefore a long-felt need for a system of measuring osmolarity that does not require expensive equipment and is suitable for personal monitoring of osmolarity or “at home” testing.

### SUMMARY OF THE INVENTION

In the best embodiment, structures (vesicles) are used that are surrounded with an integument which is permeable only to water. The vesicles' core contains a coloring agent such as a dye dissolved in water solution that has a certain, predetermined, osmotic value. When this vesicle is located in the vicinity of a liquid that has some osmotic value, one of three options can occur:
1. If osmolarity is the same (vesicle and the surrounding liquid) then no water would pass through the integument, so the vesicle does not change shape and does not burst.
2. If osmolarity is higher in the surrounding liquid then water would pass from the vesicle to the outside liquid, which will cause the vesicle to shrink
3. If osmolarity is higher in the vesicles than the surroundings, then water will enter the vesicle and will inflate the vesicle, causing the vesicle to “explode” and release its coloring agent content to the surroundings.

Such a method will allow semi-quantitative assessment of the osmolarity of a liquid in general and tear osmolarity in particular.

It is within the scope of the present invention to disclose an osmolarity measuring device for providing a semi-quantitative color measure of liquid's osmolarity, said device comprising: (a) none or more coloring agents; (b) none or more swelling agents; (c) none or more solutions; (d) a vesicle with a continuous integument, containing within said integument at least one of a group consisting of said coloring agent, said swelling agent and said solution; and (e) a support of absorbent material impregnated in at least one area with a plurality of said vesicles; wherein the apparent color of said area alters if the osmolarity of said liquid is within a pre-determined range.

It is further within the scope of the present invention that the abovementioned device has integument consisting of: (a) none or more water insoluble and permeable film-forming polymers; (b) none or more water soluble film-forming polymers; and (c) none or more permeability reducing agents.

It is further within the scope of the present invention that, for the abovementioned device with the abovementioned integument, the osmolarity is within none or more of a plurality of pre-determined ranges, each said pre-determined range identified by at least one of a plurality of integument/swelling agent combinations, with at least one said integument/swelling agent combination designed to burst at each said pre-determined value.

It is further within the scope of the present invention that, for the abovementioned device with the abovementioned integument, a change in color indicates the approximate osmolarity of the liquid, said different integument/swelling agent combinations forming at least one of a group of containing different coloring agents, containing different color removing agents, being at different locations on said support, or being impregnated on a different colored support.

It is further within the scope of the present invention that, for the abovementioned device with the abovementioned integument, it additionally comprises, within said vesicles, materials to increase the stability of said coloring agent in said solution of pre-determined osmolarity.

It is further within the scope of the present invention that, for the abovementioned device with the abovementioned integument, the coloring agent is one of a group of a dye, a pigment, a bleach, a dye decoloring agent, or a pigment decoloring agent.

It is further within the scope of the present invention that, for the abovementioned device, said integument is a liposome vesicle.

It is further within the scope of the present invention that, for the abovementioned device, said liposome vesicle is one of a group of water/oil vesicle, oil/water vesicle, water/oil/water vesicle, or oil/water/oil vesicle.

It is further within the scope of the present invention that, for the abovementioned device, said liposome vesicle is one of a group of small lipid vesicle, large lipid vesicle, very large lipid vesicle, multilamellar vesicle or multivesicular vesicle.

It is further within the scope of the present invention that, for the abovementioned device, said integument is an asymmetric liposome.

It is further within the scope of the present invention that, for the abovementioned device with the abovementioned liposome vesicle, the osmolarity is within none or more of a plurality of pre-determined ranges, each said pre-determined range identified by at least one of a plurality of integument/swelling agent combinations, with at least one said integument/swelling agent combination designed to change shape at each said pre-determined value.

It is further within the scope of the present invention that the abovementioned device is especially adapted for determining the osmolarity of tears, wherein osmolarities greater than about 270 mOs and less than about 370 mOs are determinable, in a plurality of ranges.

It is further within the scope of the present invention that the abovementioned device is especially adapted for determining the osmolarity of saliva, wherein osmolarities greater than about 0 mOs and less than about 150 mOs are determinable, in a plurality of ranges.

It is further within the scope of the present invention that, for the abovementioned device, the absorbent support is paper.

It is further within the scope of the present invention that, for the abovementioned device, at least one area of said absorbent support comprises a multi-layer structure, said multi-layer structure including at least one of a group of a non-absorbent layer, an absorbent layer, and a protective layer.

It is further within the scope of the present invention that the abovementioned device additionally comprises a chart to facilitate determination of said osmolarity by comparison of said area with said chart.

It is further within the scope of the present invention that, for the abovementioned device with a chart, said chart is one of a group of integral with said support or separate from said support.

It is further within the scope of the present invention that, for the abovementioned device with a chart, said chart is one of a group of a wall chart; a desk chart; a hand-held chart; a credit-card sized chart; or a chart on a support, either integral or attached, which enables said chart to sit in a semi-upright or upright position on a horizontal surface.

It is further within the scope of the present invention that, for the abovementioned device with a chart, said chart additionally contains at least one of a group of further information on use of the device, information on the disorder being diagnosed or under treatment, or advertising matter.

It is further within the scope of the present invention that, for the abovementioned device, the osmolarity of the liquid is indicated by a change in at least one of a group of the intensity of at least one of a plurality of colors, or the location of at least one of a plurality of colors.

It is within the scope of the present invention to disclose a method useful for determining a semi-quantitative measure of osmolarity of liquids comprising steps of (a) pre-determining a plurality of osmolarity values; (b) choosing an absorbent support; (c) choosing at least one of: (i) a coloring agent, (ii)a color for said absorbent support, or (iii) at least one color for at least one area on said absorbent support; (d) creating vesicles with a continuous integument which will alter shape at at least one of said plurality of osmolarity ranges containing at least one of: (i) a coloring agent, (ii) a swelling agent, or (iii) a solution; (e) impregnating said absorbent support with said vesicles; (f) exposing said impregnated support to said liquid; wherein the apparent color of, said area alters if the osmolarity of said liquid is within a pre-determined range.

It is further within the scope of the present invention that, for the abovementioned method, said integument consists of: (a) none or more water insoluble and permeable film-forming polymers; (b) none or more water soluble film-forming polymers; and (c) none or more permeability reducing agents.

It is further within the scope of the present invention that, for the abovementioned method with the abovementioned integument, each of said pre-determined ranges is identified by at least one of a plurality of integument/swelling agent combinations, with at least one said integument/swelling agent combination designed to burst at each said pre-determined value.

It is further within the scope of the present invention that, for the abovementioned method with the abovementioned integument, the color indicates the osmolarity of the liquid, said different integument/swelling agent combinations forming at least one of a group of containing different coloring agents, containing different color removing agents, being at different locations on said support, or being impregnated on a different colored support.

It is further within the scope of the present invention that, for the abovementioned method with the abovementioned integument, said vesicles additionally contain materials to increase the stability of said coloring agent in said solution of pre-determined osmolarity.

It is further within the scope of the present invention that, for the abovementioned method with the abovementioned integument, the coloring agent is one of a group of a dye, a pigment, a bleach, a dye decoloring agent, or a pigment decoloring agent.

It is further within the scope of the present invention that, for the abovementioned method, said integument is a liposome vesicle.

It is further within the scope of the present invention that, for the abovementioned method with a liposome vesicle, said liposome vesicle is one of a group of water/oil vesicle, oil/water vesicle, water/oil/water vesicle, or oil/water/oil vesicle.

It is further within the scope of the present invention that, for the abovementioned method with a liposome vesicle, said liposome vesicle is one of a group of small lipid vesicle, large lipid vesicle, very large lipid vesicle, multilamellar vesicle or multivesicular vesicle.

It is further within the scope of the present invention that, for the abovementioned method with a liposome vesicle, said integument is an asymmetric liposome.

It is further within the scope of the present invention that, for the abovementioned method with a liposome vesicle, the osmolarity is within none or more of a plurality of pre-determined ranges, each said pre-determined range identified by at least one of a plurality of integument/swelling agent combinations, with at least one said integument/swelling agent combination designed to change shape at each said pre-determined value.

It is further within the scope of the present invention that, for the abovementioned method, the absorbent support is paper.

It is further within the scope of the present invention that, for the abovementioned method, at least one area of said absorbent support comprises a multi-layer structure, said multi-layer structure including at least one of a group of a non-absorbent layer, absorbent layer, and protective layer.

It is further within the scope of the present invention that the abovementioned method is especially adapted for determining the osmolarity of tears, wherein osmolarities greater than about 270 mOs and less than about 370 mOs are determinable, in a plurality of ranges.

It is further within the scope of the present invention that the abovementioned method is especially adapted for determining the osmolarity of saliva, wherein osmolarities greater than about 0 mOs and less than about 150 mOs are determinable, in a plurality of ranges.

It is further within the scope of the present invention that, for the abovementioned method, determination of said osmolarity is facilitated by comparison of said area with a chart.

It is further within the scope of the present invention that, for the abovementioned method including a chart, said chart is one of a group of integral with said support or separate from said support.

It is further within the scope of the present invention that, for the abovementioned method including a chart, said chart is one of a group of a wall chart; a desk chart; a hand-held chart; a credit-card sized chart; or a chart on a support, either integral or attached, which enables said chart to sit in a semi-upright or upright position on a horizontal surface.

It is further within the scope of the present invention that, for the abovementioned method including a chart, said chart additionally contains at least one of a group of further information on use of the device, information on the disorder being diagnosed or under treatment, or advertising matter.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates one embodiment of the device, where the square 130 changes color if the osmolarity is within a predetermined range.

FIG. 2 schematically illustrates another embodiment of the device, where the squares 230 change color within predetermined ranges, said ranges differing at at least one end, and said squares being arranged longitudinally along the strip 210.

FIG. 3 schematically illustrates another embodiment of the device, where the squares 230 change color within predetermined ranges, said ranges differing at at least one end, and said squares being arranged laterally across the strip 310.

### DETAILED DESCRIPTION OF THE PREFERRED EMBODIEMNTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for quickly assessing the osmolarity of a liquid in a semi-quantitative manner.

The term ‘integument’ hereinafter refers to an outer covering.

The term ‘vesicle’ hereinafter refers to a continuous integument containing a liquid, where said integument may also contain a coloring agent, a swelling agent, and possibly inert ingredients.

The term ‘burst’ hereinafter refers to the continuous integument surrounding a vesicle breaking, thereby releasing its contents.

The term ‘osmolarity’ hereinafter refers to the amount of material dissolved in a solute. In general, three terms are used to define the amount of material dissolved in a solute. Where molarity is the concentration of a compound in a unit volume of solute, osmolarity is the concentration of compound particles in a unit volume of solute and osmolality is the concentration of compound particles in a unit mass of solute. If the compound does not dissociate, the molarity and the osmolarity are equal.

Osmolarity can be calculated from osmolality by the following equation: [Page 158 in: Martin, Alfred N.; Patrick J Sinko (2006). Martin's physical pharmacy and pharmaceutical sciences: physical chemical and biopharmaceutical principles in the pharmaceutical sciences. Phila: Lippincott Williams and Wilkins. ISBN 0-7817-5027-X.]

Osmolarity=osmolality*(ρₛₒₗ−cₐ)

where:

ρₛₒₗ is the density of the solution in g/ml and

cₐ is the (anhydrous) solute concentration in g/ml.

Osmolality is independent of temperature, while osmolarity is dependent on temperature via the dependence of the density of the solute on temperature. In practice, the variation of osmolarity with temperature is small in the temperature ranges likely to be encountered in practice, so “osmolarity” will be used consistently herein.

In one embodiment of the device, the integument is composed of a multilayer membrane, such as is taught in U.S. Pat. No. 5,260,069. In this Patent, a plurality of populations of vesicles is provided within a unit dosage form such as a capsule or tablet. The vesicles are composed of a core containing the drug and a swelling agent which expands in volume when exposed to water. The core is enclosed within an integument which is permeable to water. The integument is composed of a water insoluble and permeable film-forming polymer, a water soluble film-forming polymer and a permeability reducing agent. When the unit dose releases the vesicles into the digestive tract, water diffuses through the coating and into the core. As water is taken up by the swelling agent, the core expands, exerting force on the coating until it bursts, releasing the drug. The permeability reducing agent reduces the rate at which water reaches the swelling agent, thereby delaying release time. The water soluble polymer dissolves, weakening the coating so that it bursts sooner. By varying the proportions of the three coating ingredients and/or coating thickness from one vesicle population to another, the release timing of the vesicles can be very effectively controlled.

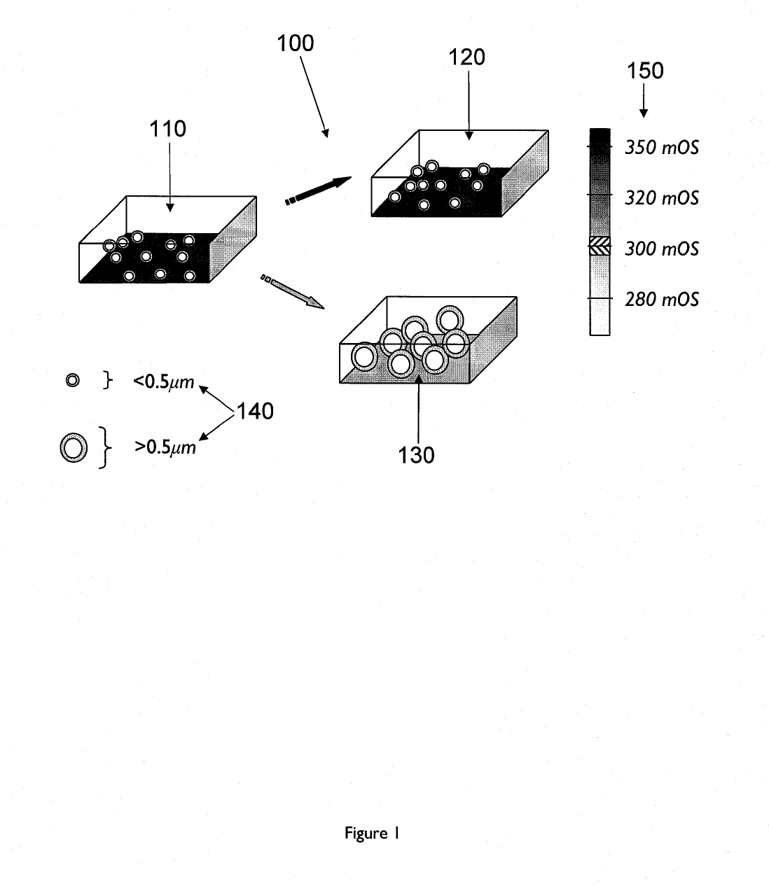

In another embodiment, the integument is formed from liposome microspheres. Liposome microspheres used for drug delivery are taught in many patents, including RU2211693C2, CN1788723A, and CN101485665A. With reference to FIG. 1, an area of the support impregnated with vesicles (100) is shown, with vesicles in it. Vesicles with diameters of less than 0.5 μm (140) will allow light to pass through them and be reflected from the underlying support, while vesicles with diameters greater than 0.5 μm will absorb light so that the underlying color of the support will be hidden. In the dry state (110) the vesicles are of medium size; some of the color of the underlying support will be seen. If exposed to a liquid of high osmolarity (120), the vesicles will shrink and the apparent color of the underlying support will become more intense. If exposed to a liquid of low osmolarity (130), the vesicles expand, absorb visible light, and the apparent color of the underlying support will become less intense. The chart (150) at the right hand edge of FIG. 1 shows the apparent intensity of color as a function of osmolarity. The diagonally striped areas on the chart (150) mark the limits of normal osmolarity. Such a chart could be provided imprinted on a device to measure, osmolarity according to this invention, in order to improve the accuracy with which the device is read.

In an embodiment of the device, to measure the osmolarity of the tear film of the eye to assess or diagnose dry eye syndrome, tears would be absorbed from the eye, in the same manner as is commonly done in the Schirmer test, on to an absorbent support such as abosorbent paper where at least one area of the absorbent support is impregnated with vesicles of a multilayer polymer produced in a manner similar to U.S. Pat. No. 5,260,069 but where said vesicles contains a coloring agent such as a dye in solution at an osmolarity of 300 mOs.

If the tear osmolarity is relatively high (e.g. 350 mOs) then the vesicles will shrink and the absorbing paper will remain its original color. If the tear film osmolarity is relatively low (e.g. 280 mOs), then the vesicles will burst and the coloring matter will be released, hence leaving color (e.g. red) on the absorbent support. The lower the osmolarity, the more intense the color will be (the redder the support will appear).

In another embodiment of the device, the integument consists of a liposome vesicle, and there are sufficient vesicles to mask at least most of the underlying color of the area of the support impregnated with vesicles. If the osmolarity of the tear film is relatively high (e.g. 350 mOs), then the vesicles will shrink, allowing the underlying color to be seen. If the osmolarity of the tear film is relatively low (a e.g. 280 mOs), then the liposomes will swell and the underlying color will be masked.

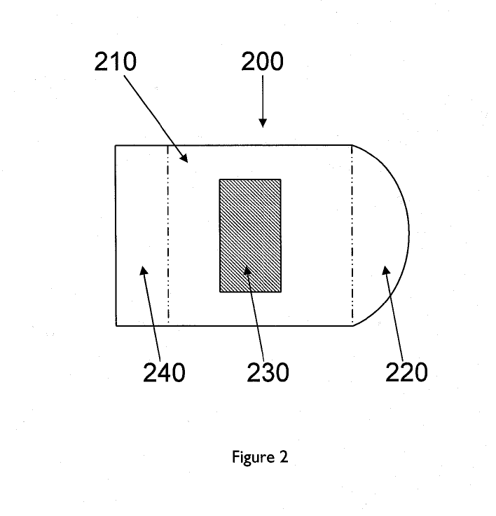

With reference to FIG. 2, the device (200) consists of a strip of absorbent material (210) with a rounded distal end (220) which is inserted into the lower conjunctival fornix, and a proximal end (240) by which the strip (210) may be held. Between the proximal and distal ends is an area (230) containing vesicles of a colorant in solution at a pre-determined osmolarity. If the osmolarity of the liquid is less than a predetermined osmolarity, the vesicles burst, coloring the strip in the region of said area.

In an embodiment of the design of FIG. 2, the pre-determined osmolarity is 300 mOs. The strip is white or undyed absorbent paper, the integument is a multilayer polymer and the coloring matter is a red dye, so that, if the osmolarity of the liquid is below 300 mOs, the paper will be colored red (or any other convenient color).

For diagnosis and/or treatment of conjunctivitis sicca, said red color will indicate a normal osmolarity of the tear liquid.

In another embodiment of the device taught above, the paper or the area under the vesicles is red (or some other convenient color), the integument is of liposomes and the pre-determined osmolarity is 300 mOs. The region of said area appears pink to white if tear osmolarity is within the normal range.

In another embodiment of the devices taught above, a piece of red (or some other convenient color) film is embedded in or inserted into or adherent on the paper and the colorant is a white pigment or dye. If the osmolarity of the liquid is below 300 mOs, the region of the film appears pink to white if tear osmolarity is within the normal range.

In another embodiment of the device in FIG. 2, vesicles impregnated within said area burst at a plurality of osmolarities, so that the more intense the color, the lower the osmolarity of the liquid. As an illustrative example, the osmolarities at which at least one population of vesicles will burst are 300 mOs, 320 mOs and 340 mOs.

In another embodiment of the above device, the paper is colored or contains a colored area, as described above, and the vesicles have a liposome integument, so that the paler the area appears, the lower the osmolarity of the liquid.

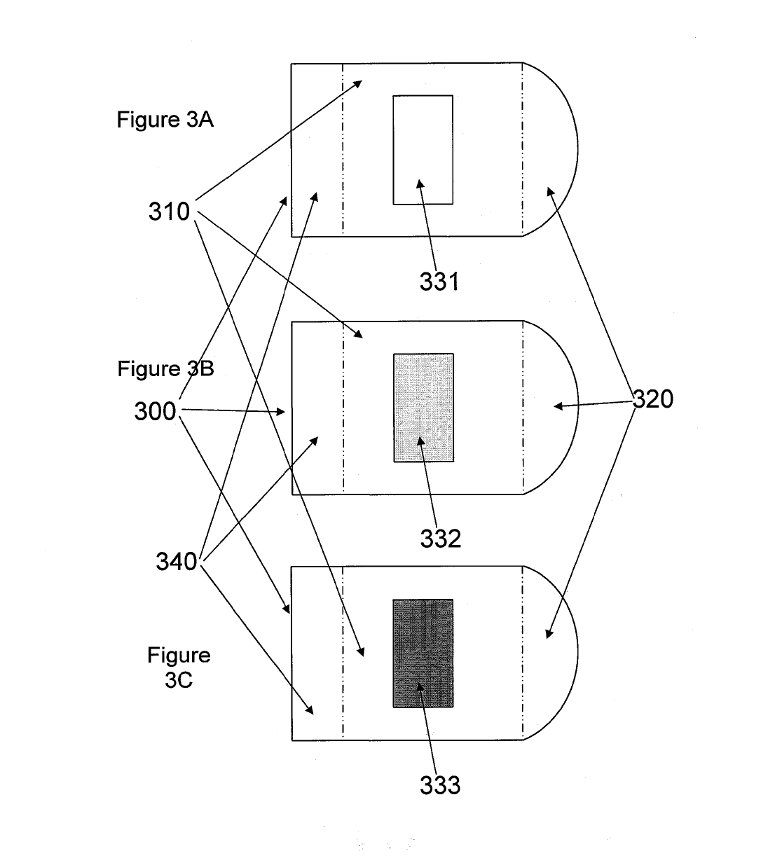

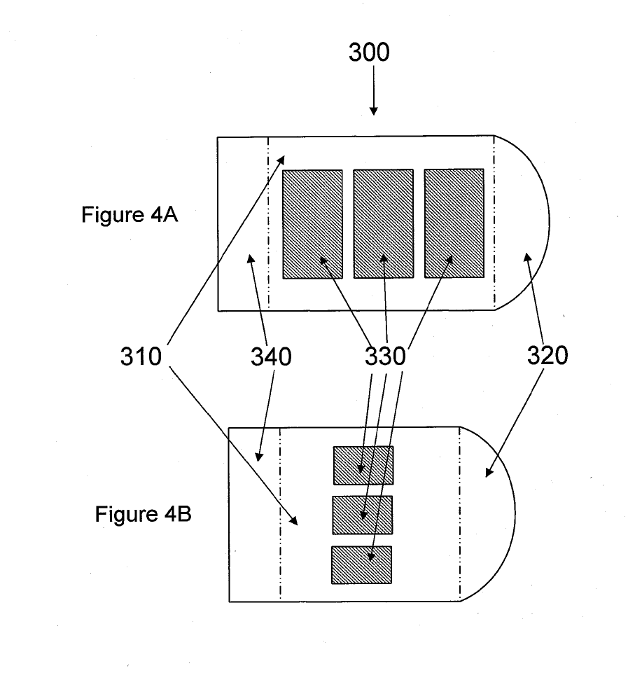

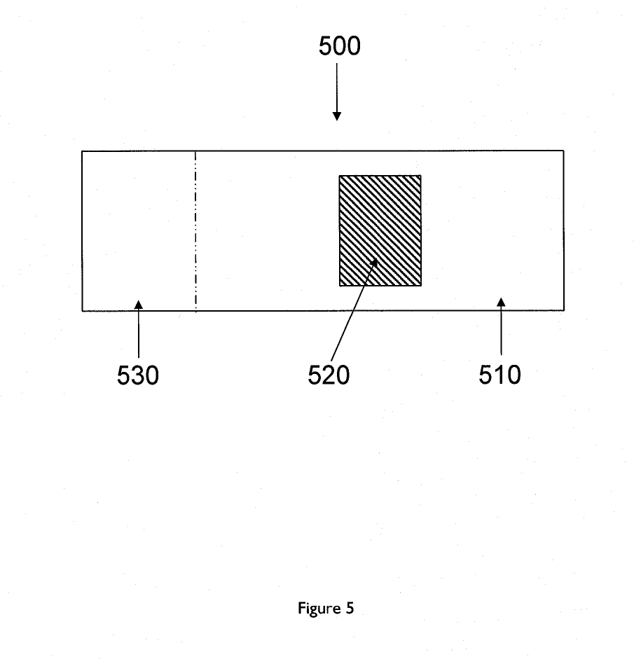

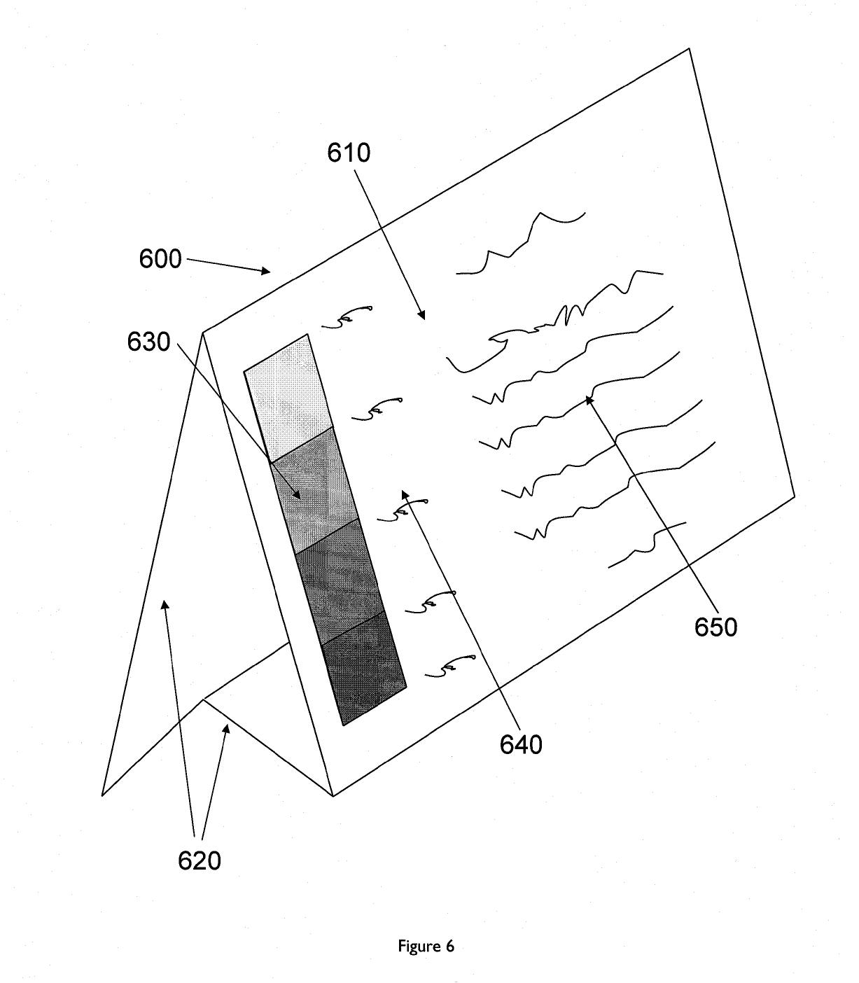

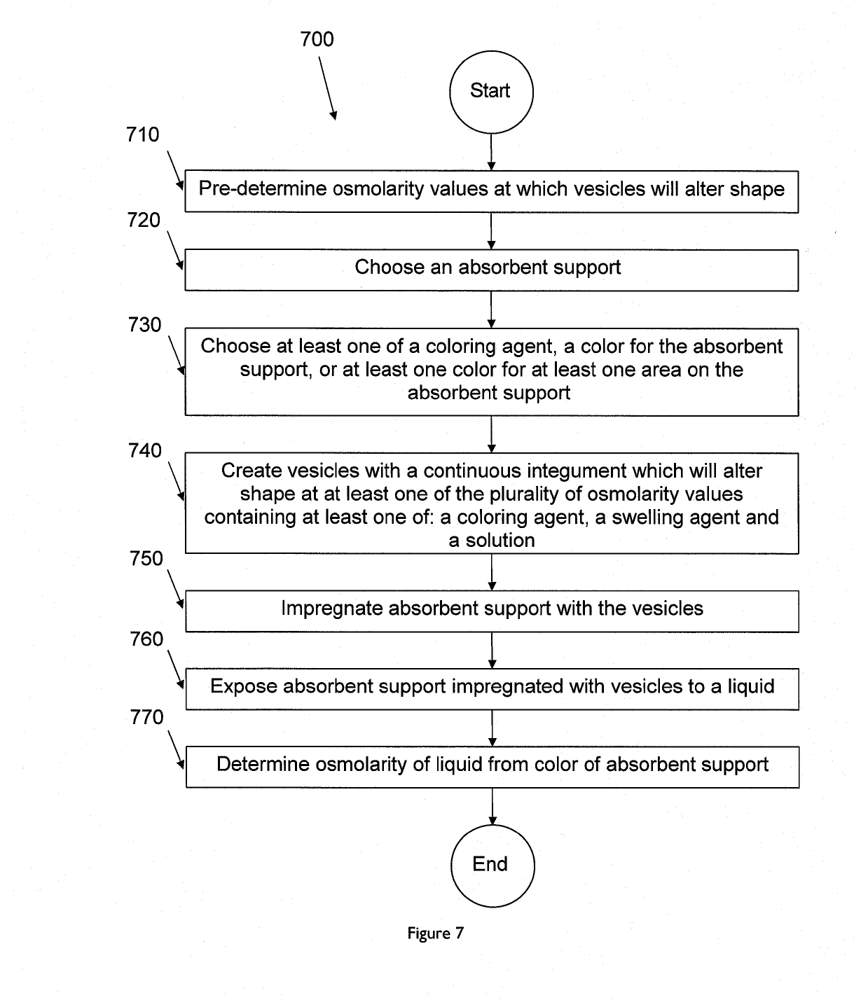

With reference to FIG. 3, as an illustrative example, the device of the above embodiment is shown after exposure to tear fluid of different osmolarities. In this FIG. (300), the strip (310) has a rounded distal end (320), and a proximal end (340). For FIG. 3A, the osmolarity is high and there is little change in the color of the impregnated area (331). For FIG. 3B, the osmolarity is moderate and there is some change in the color of said area (332). For FIG. 3C, the osmolarity is low and there is a large change in the color of the impregnated area (333).

In another embodiment of the device (FIG. 4) the device (400) consists of a strip (410) with rounded distal end (420) which is inserted into the lower conjunctival fornix, and a proximal end (440) by which the strip (410) may be held. Between the proximal and distal ends are three areas (430), each containing vesicles designed to burst at different pre-determined osmolarities. In FIG. 4A, the areas (430) are arranged longitudinally; in FIG. 4B, they (430) are arranged laterally.

In this particular embodiment, the pre-determined osmolarities are 300 mOs, 320mOs and 340 mOs.

In another embodiment of the above devices, the paper or an area on the paper or the package or envelope containing the vesicles is colored and the vesicles contain a bleach or other decoloring agent to which the coloring agent is sensitive so that, when the vesicles burst, the color is removed from the paper or envelope so that lack of color indicates osmolarity lower than the pre-set value or values.

In another embodiment of the device (FIG. 5), intended for measuring the osmolarity of saliva, the device (500) consists of a strip of absorbent material (510) with a proximal end (530) which is placed in contact with saliva in the mouth, either against the inside of the cheek, or on the tongue, or under the tongue. The saliva then migrates to in area impregnated with vesicles (520), exposing the vesicles in it to the saliva. In this embodiment, the area (520) contains vesicles with pre-determined osmolarities of 40 mOs, 60 mOs, 80 mOs and 100 mOs.

In another embodiment of the above devices, a chart indicating the correlation between color and osmolarity is imprinted on or adherent to or otherwise visible on the paper strip.

In another embodiment of the above devices, a separate chart indicating the correlation between color and osmolarity provided. Said chart may be in the form of, but is not limited to, a wall chart; a hand-held chart; a desk chart; a credit-card sized chart convenient for carrying in a wallet, whether or not it is plastic or plastic covered like a credit card; or a chart with a stand, either integral or attached, designed to sit on a desk or table (FIG. 6).

In FIG. 6, the chart with a stand (600) has a front part (610) and a back support part (620). On the front part (610), there is a color comparison chart (630) with sufficient labeling (640) to enable semi-quantitative determination of osmolarity by comparison of the strip with the color comparison chart. The front part (610) also includes other information (650), which may include at least text or graphic material or both as described below.

Any of said charts could contain, in additional to the correlation chart, none or more of: supplemental information about the use of the device; if the device is used for diagnosis of a disorder or during treatment for a disorder, supplemental information about said disorder or said treatment; a calendar; or advertising material.

With reference to FIG. 7, a flow chart (700) of the method is taught. The osmolarity value(s) of interest are determined (710) and the dye or dyes chosen (710) if a multilayer membrane is to be used for the vesicles. For each of the osmolarity values of interest, a combination of swelling agent, solution of pre-determined osmolarity, and integument are established (730) such that the integument will burst when in contact with a liquid of that osmolarity value. A strip of absorbent paper is then impregnated with the vesicles (740), in such a manner that, on examination, it is clear which of the plurality of types of vesicle have burst. If there is to be more than one such value, this may be done by having a single area impreganted with the vesicles, so that the more intense the color, the higher the osmolarity, or by having several areas, either the same color or different colors, so that the location of the colored areas establishes the osmolarity. The absorbent paper is then exposed to the liquid of interest (750) and the osmolarity of the liquid is determinable from the resulting colors (760).

With the ability to determine a threshold for osmolarity, one can determine in a semi-quantitative manner the osmolarity of a liquid.

1. An osmolarity measuring device for providing a semi-quantitative color measure of liquid's osmolarity, said device comprising:
a. one or more coloring agents;
b. one or more swelling agents;
c. one or more solutions;
d. a vesicle with a continuous integument, containing within said integument at least one of a group consisting of said coloring agent, said swelling agent and said solution; and
e. a support of absorbent material impregnated in at least one area with a plurality of said vesicles;
wherein the apparent color of said area alters if the osmolarity of said liquid is within a pre-determined range.

2. The device of claim 1 wherein said integument consists of:
a. one or more water insoluble and permeable film-forming polymers;
b. one or more water soluble film-forming polymers; and
c. one or more permeability reducing agents.

3. The device of claim 2 wherein the osmolarity is within one or more of a plurality of pre-determined ranges, each said pre-determined range identified by at least one of a plurality of integument/swelling agent combinations, with at least one said integument/swelling agent combination designed to burst at each said pre-determined value.

4. The device of claim 2 wherein a change in color indicates the approximate osmolarity of the liquid, said different integument/swelling agent combinations forming at least one of a group of containing different coloring agents, containing different color removing agents, being at different locations on said support, or being impregnated on a different colored support.

5. The device of claim 2 additionally comprising, within said vesicles, materials to increase the stability of said coloring agent in said solution of pre-determined osmolarity.

6. The device of claim 2 wherein the coloring agent is one of a group of a dye, a pigment, a bleach, a dye decoloring agent, or a pigment decoloring agent.

7. The device of claim 1 wherein said integument is a liposome vesicle.

8. The device of claim 7 wherein said liposome vesicle is one of a group of water/oil vesicle, oil/water vesicle, water/oil/water vesicle, or oil/water/oil vesicle.

9. The device of claim 7 wherein said liposome vesicle is one of a group of small lipid vesicle, large lipid vesicle, very large lipid vesicle, multilamellar vesicle or multivesicular vesicle.

10. The device of claim 7 wherein said integument is an asymmetric liposome.

11. The device of claim 7 wherein the osmolarity is within sue or more of a plurality of pre-determined ranges, each said pre-determined range identified by at least one of a plurality of integument/swelling agent combinations, with at least one said integument/swelling agent combination designed to change shape at each said pre-determined value.

12. The device of claim 1 especially adapted for determining the osmolarity of tears, wherein osmolarities greater than about 270 mOs and less than about 370 mOs are determinable, in a plurality of ranges.

13. The device of claim 1 especially adapted for determining the osmolarity of saliva, wherein osmolarities greater than about 0 mOs and less than about 150 mOs are determinable, in a plurality of ranges.

14. The device of claim 1 wherein the absorbent support is paper.

15. The device of claim 1 wherein at least one area of said absorbent support comprises a multi-layer structure, said multi-layer structure including at least one of a group of a non-absorbent layer, an absorbent layer, and a protective layer.

16. The device of claim 1 additionally comprising a chart to facilitate determination of said osmolarity by comparison of said area with said chart.

17. The device of claim 16 wherein said chart is one of a group of integral with said support or separate from said support.

18. The device of claim 16 wherein said chart is one of a group of a wall chart;
a desk chart; a hand-held chart; a credit-card sized chart; or a chart on a support, either integral or attached, which enables said chart to sit in a semi-upright or upright position on a horizontal surface.

19. The device of claim 16 wherein said chart additionally contains at least one of a group of further information on use of the device, information on the disorder being diagnosed or under treatment, or advertising matter.

20. The device of claim 1 wherein the osmolarity of the liquid is indicated by a change in at least one of a group of the intensity of at least one of a plurality of colors, or the location of at least one of a plurality of colors.

21. A method useful for determining a semi-quantitative measure of osmolarity of liquids comprising steps of
a. pre-determining a plurality of osmolarity values;
b. choosing an absorbent support;
c. choosing at least one of:
i. a coloring agent,
ii. a color for said absorbent support, or
iii. at least one color for at least one area on said absorbent support;
d. creating vesicles with a continuous integument which will alter shape at at least one of said plurality of osmolarity ranges containing at least one of:
i. a coloring agent,
ii. a swelling agent, or
iii. a solution;
e. impregnating said absorbent support with said vesicles;
f. exposing said impregnated support to said liquid;
wherein the apparent color of said area alters if the osmolarity of said liquid is within a pre-determined range.

22. The method of claim 21 wherein said integument consists of:
a. one or more water insoluble and permeable film-forming polymers;
b. one or more water soluble film-forming polymers; and
c. one or more permeability reducing agents.

23. The method of claim 22 wherein each of said pre-determined ranges is identified by at least one of a plurality of integument/swelling agent combinations, with at least one said integument/swelling agent combination designed to burst at each said pre-determined value.

24. The method of claim 22 wherein the color indicates the osmolarity of the liquid, said different integument/swelling agent combinations forming at least one of a group of containing different coloring agents, containing different color removing agents, being at different locations on said support, or being impregnated on a different colored support.

25. The method of claim 22 wherein said vesicles additionally contain materials to increase the stability of said coloring agent in said solution of pre-determined osmolarity.

26. The method of claim 22 wherein the coloring agent is one of a group of a dye, a pigment, a bleach, a dye decoloring agent, or a pigment decoloring agent.

27. The method of claim 21 wherein said integument is a liposome vesicle.

28. The method of claim 27 wherein said liposome vesicle is one of a group of water/oil vesicle, oil/water vesicle, water/oil/water vesicle, or oil/water/oil vesicle.

29. The method of claim 27 wherein said liposome vesicle is one of a group of small lipid vesicle, large lipid vesicle, very large lipid vesicle, multilamellar vesicle or multivesicular vesicle.

30. The method of claim 27 wherein said integument is an asymmetric liposome.

31. The method of claim 27 wherein the osmolarity is within one or more of a plurality of pre-determined ranges, each said pre-determined range identified by at least one of a plurality of integument/swelling agent combinations, with at least one said integument/swelling agent combination designed to change shape at each said pre-determined value.

32. The method of claim 21 wherein the absorbent support is paper.

33. The method of claim 21 wherein at least one area of said absorbent support comprises a multi-layer structure, said multi-layer structure including at least one of a group of a non-absorbent layer, absorbent layer, and protective layer.

34. The method of claim 21 especially adapted for determining the osmolarity of tears, wherein osmolarities greater than about 270 mOs and less than about 370 mOs are determinable, in a plurality of ranges.

35. The method of claim 21 especially adapted for determining the osmolarity of saliva, wherein osmolarities greater than about 0 mOs and less than about 150 mOs are determinable, in a plurality of ranges.

36. The method of claim 21 wherein determination of said osmolarity is facilitated by comparison of said area with a chart.

37. The method of claim 21 wherein said chart is one of a group of integral with said support or separate from said support.

38. The method of claim 21 wherein said chart is one of a group of a wall chart; a desk chart; a hand-held chart; a credit-card sized chart; or a chart on a support, either integral or attached, which enables said chart to sit in a semi-upright or upright position on a horizontal surface.

39. The method of claim 21 wherein said chart additionally contains at least one of a group of further information on use of the device, information on the disorder being diagnosed or under treatment, or advertising matter.
